(19) United States
(12) Patent Application Publication
(10) Pub. No.: US 2006/0062862 A1
(43) Pub. Date: Mar. 23, 2006

(54) Cholesterol-reducing agent made of dietary fibre and cholesterol-reducing substances

(76) Inventors: Bernd Haber, Mainz (DE); Hans-Ulrich Ter Meer, Frankfurt (DE); Stephan Hausmanns, Wiesbaden (DE)

Correspondence Address: PROPAT, L.L.C., 425-C SOUTH SHARON AMITY ROAD, CHARLOTTE, NC, 28211-2841, US

(21) Appl. No.: 10/521,503

(22) PCT Filed: Jul. 15, 2003
(86) PCT No.: PCT/EP03/07624

§ 371 Date: Jan. 18, 2005

(30) Foreign Application Priority Data
Jul. 23, 2002 (DE) 102 33 342.4
Jan. 31, 2003 (DE) 103 03 900.7
May 9, 2003 (DE) 103 20 983.2

(51) Int. Cl. A61K 36/899; A61K 36/48; A61K 31/401; A61K 31/366; A61K 31/455; A61K 31/225

(52) U.S. Cl. 424/750; 424/757; 514/423; 514/460; 514/548; 514/171; 514/355

(57) ABSTRACT

This invention relates to cholesterol reducing agents made of dietary fiber and at least one cholesterol-reducing active ingredient. This invention also relates to methods for the production of the agents and uses thereof.

The invention relates to cholesterol-reducing agents made of dietary fiber and to at least one cholesterol-reducing active ingredient. The invention further relates to a method for producing such agents and use thereof.

In the context of an unbalanced diet, in broad sections of the population, an increased content of blood fat values, in particular blood cholesterol values, is found. A cholesterol value greater than 200 mg/dl, in particular LDL cholesterol values greater than 130 mg/dl, is considered one of the main risk factors of cardiovascular disorders. Therefore, therapeutic treatment in the case of significantly increased cholesterol values, in particular LDL cholesterol, is essential. A number of approaches of solutions have been previously described for this. In addition to the usually only slightly active changeover of lifestyle and dietary habits, a number of special active ingredients have been developed which intervene in different ways in the absorption and metabolism of cholesterol. These are, inter alia, pharmacologically active substances, such as statins (inter alia U.S. Pat. No. 4,231,938, U.S. Pat. No. 4,444,784, U.S. Pat. No. 4,346,227), inhibitors of bile acid uptake (inter alia U.S. Pat. No. 5,998,400, U.S. Pat. No. 6,277,831, U.S. Pat. No. 6,221,897) or bile acid sequestrants (inter alia U.S. Pat. No. 4,027,009). All of these active ingredients must be taken under medical direction and supervision.

Among the active ingredients can also be included cholesterol-reducing compounds isolated from plant sources. Here, especially, the cholesterol-reducing action of a group of plant sterols, in particular phytosterols, phytostanols, and the esters of said classes of compound (inter alia WO 96/38047, WO 99/56558, U.S. Pat. No. 6,087,353) may be mentioned. The latter, especially, however, are not suitable for being taken by all sections of the population (for example exclusions for pregnant women or infants) and are frequently limited in their application. Further natural cholesterol-lowering active ingredients also include extracts from further plant sources, for example artichoke extracts, tocotrienol-rich extracts, garlic or guglipid extracts as are mentioned, for example, in the publications EP-A-1 238 590 or IN-A-166998.

Soy protein-containing products also display cholesterol-reducing properties (Anderson J W, Johnstone B M, Cook-Newell M E, Metaanalysis of the effects of soy protein intake on serum lipids, NEW ENGLAND JOURNAL OF MEDICINE, 1995, 333(5), 276-82).

On the other hand, there are food components which have shown repeatedly that, in the case of sufficient intake, can significantly reduce the risk of cardiovascular disorders, in particular by reducing elevated cholesterol levels. For dietary fiber as typical food component, it is generally known that a high dietary fiber consumption in the diet is, compared with a low-dietary-fiber diet, beneficially associated with a lower risk of cardiovascular disorders (Jacobs et al. 1998: Am J Clin Nutr. 68: 248-257; Wolk et al. 1999; JAMA 2281; 1998-2004). In addition to whole-grain cereals such as wheat, oats, barley, rye and also cereal brans such as oat bran, rice bran, wheat bran, soy bran, etc., which are generally rich in dietary fiber, other dietary fibers can also make a beneficial contribution to reducing the cardiovascular risk and elevated cholesterol levels. For instance, a number of water-soluble dietary fibers, for example β-glucan (from oats or barley), psyllium, pectin or guar gum exhibit a reducing action on the blood cholesterol level (Brown et al. 1999; Am. J. Clin. Nutr. 69: 30-42).

Furthermore, as food components, levans are known which can significantly reduce serum cholesterol values, selectively that is to say without reducing the triglycerol or glucose level in the serum (Yamamoto et al. 1999, J. Nutr. Biochem. 10, 13-18, and Yamamoto et al. 2000, Hydrocolloids Part 2, Fundamentals and Application in Food, Biology and Medicine, Elsevier, 2000, 399-404).

Furthermore, as food components, water-insoluble carob fibers are known, preferably those produced by a process according to EP-A-0 616 780, which can significantly reduce serum cholesterol values, in particular the LDL cholesterol (Zunft et al. 2001; Adv. In Ther. 18: 230-36). The HDL value remains constant in this, so that the important LDL/HDL ratio is shifted toward the “good cholesterol”, and thus the risk of arteriosclerosis decreases.

The effects achievable, in the case of food components, however, are markedly below those which are achieved using therapeutic active ingredients, and thus far lower than desirable. Even if a dietary-fiber-enriched diet can thus make a contribution to controlling the cholesterol level, in many cases, in particular in the case of very high cholesterol levels (total cholesterol>300 mg/dl) is insufficient for lowering which persists.

A synergistic cholesterol-reducing interaction between food components, in particular dietary fibers such as carob fibers or levans, and active ingredients is not known. Within the group of food components, for example, even an antagonistic action in the case of soluble dietary fibers of carob bean meal with water-insoluble fibers of the carob fruit flesh have been described (Peres-Olleros et al. 1999; J. Sci. Food Agric. 79, 173-178).

The purely pharmacological cholesterol-lowering compounds have the disadvantage that to achieve the therapeutic goals, in some cases considerable concentrations need to be used. Unwanted, sometimes life-threatening side effects can occur, also in combination with other therapeutic agents. Combination therapies to increase the efficacy with various cholesterol-reducing active ingredients, or else other therapeutic agents, for example for cardiovascular disorders, cannot always be used because of various dangerous contraindications. For instance, combinations of fibrates with statins exhibit an elevated risk of myopathy syndromes which, in the case of combinations of cerivastatin with gemfibrozil can even be fatal.

Furthermore, saturation effects are known which have the effect that, with increased intake of the active ingredient, only slightly additional reductions of the cholesterol level are achieved. A further disadvantage is the high costs which occur in the case of long-term therapies using the usually very expensive pharmacological cholesterol-reducing compounds.

In the case of the cholesterol-reducing compounds isolated from plant sources (for example phytosterols), there are quantitative limitations to avoid unwanted side effects.

In WO 03/018024, combination preparations of a dietary fiber and 1,4-benzothiepine 1,1-dioxide derivatives and in WO 03/018059, combination preparations of a dietary fiber and aryl-substituted propanolamine derivatives are proposed.

There is still, therefore, a requirement for cholesterol-reducing agents which, with the same or even improved activity, reduce the amounts of the respective active ingredient administered and thus reduce any side effects and costs, in particular of long-term therapies.

This object is achieved by cholesterol-reducing agents made of at least one dietary fiber and at least one cholesterol-reducing active ingredient.

Dietary fibers in the meaning of the invention are taken to mean constituents of the plant cells and/or isolated natural substances, or substances produced by technological processes, for example extracts, which are not broken down by the human enzyme system in the small intestine to give absorbable components. However, they can be partially or completely fermented by the large-intestine flora. The dietary fibers can be selected, for example, from one or more of the following substances: whole-grain cereals (wheat, oat, barley, rye), oat bran (β-glucan), rice bran, corn bran, barley, psyllium husk, guar, carob beans, tragacanth, pectin, inulin, indigestible oligosaccharides, carob fruit flesh, linseed, dietary soy fiber, soy bran, dextrins, arabinoxylans, arabinogalactans and levans.

Preferred dietary fibers within the meaning of the invention are carob fibers and levans.

Dietary fibers within the meaning of the invention which are preferred in particular are carob fibers, with those being very particularly preferred, which are characterized by a high content of insoluble dietary fibers, but also polyphenols. The content of total dietary fibers of the carob fiber, determined as specified by AOAC method 985.29, is at least 30% by weight, preferably at least 60% by weight, but particularly preferably at least 80% by weight (in each case based on the dry mass). Their content of water-insoluble dietary fiber, determined by AOAC method 991.42, is at least 25% by weight, preferably at least 50% by weight, but particularly preferably at least 70% by weight (in each case based on dry mass). The dietary fiber is produced in such a manner that the fruit flesh which has been freed from carob beans is, in a continuous extraction process, predominantly separated from the water-soluble carob components, and the resultant residue is dried, ground, and, if appropriate, sieved, with particle sizes of <1000 μm, preferably <500 μm, and in particular preferably of <200 μm, being obtained. Particular preference is given to the method of EP-A-0 616 780. The resultant preparations exhibit a pronounced hypocholesterolemic action, and can be used to enrich foods.

Levan within the meaning of the invention is taken to mean a beta-2,6,-polyfructan which, according to isolation or production, can have additional beta-2,1-fructofuranosyl bonds and molecular weights (M_{w}) between 10³ and 10⁷. The dietary fiber can be produced, for example, in such a manner that sucrose is converted to levan in a biocatalytic reaction using an enzyme having the catalytic activity of a levan sucrase and is then filtered, washed and dried. In the reaction, levan sucrase can be used alone or together with further glycosyl transferases to produce branched levans. Preference is given to the method according to WO 99/40217 or WO 00/31287. Particularly preferably, the production process is controlled in such a manner that particularly long-chain levans having high molar masses>5×10⁵ are produced. The preparations thus isolated exhibit a pronounced hypocholesterolemic action and can be used to enrich foods.

Cholesterol-reducing active ingredients within the meaning of the invention are taken to mean active ingredients which can reduce an elevated cholesterol level (>200 mg/dl), in particular LDL cholesterol level>130 mg/dl. These are distinguished in that they specifically influence certain metabolic processes and as a result lead in a secondary manner to a reduction of the LDL cholesterol and the total cholesterol (generally between 10 and 55%).

The active ingredients within the meaning of the invention comprise cholesterol-reducing substances of the group of the statins, the bile acid resorption inhibitors and bile acid sequestrants, cholesterol absorption inhibitors, fibrates, nicotinic acid derivatives, and also the group of phytosterols and plant stanols and also cholesterol-reducing plant extracts, for example from artichokes or guglipid, and also soy protein-containing products.

The active group statins is taken to mean compounds such as lovastatin [see formula 1 below] (e.g. U.S. Pat. No. 4,231,938), paravastatin (e.g. U.S. Pat. No. 4,346,227), simvastatin [see formula 2 below] (e.g. U.S. Pat. No. 4,444,784), fluvastatin (e.g. U.S. Pat. No. 5,354,772), atorvastatin (e.g. U.S. Pat. No. 5,273,995) or cerivastatin (e.g. U.S. Pat. No. 5,177,080) which act specifically in the liver via inhibition of cholesterol synthase (HMG CoA reductase inhibitors). These active substances have been described many times and are widely used as drugs and for therapy (e.g. U.S. Pat. No. 6,180,660) for cholesterol reduction.

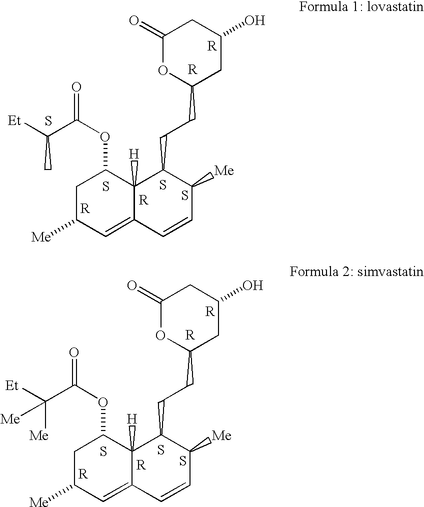

Inhibitors of bile acid resorption within the meaning of the invention are taken to mean substances which prevent the reuptake of bile acids in the intestine/ileum via a receptor-mediated process. These are, in particular, benzothiazepine derivatives (U.S. Pat. No. 5,998,400, U.S. Pat. No. 6,277,831), benzothiepine 1,1-dioxide derivatives (U.S. Pat. No. 6,221,897, WO 97/33882), in particular compounds according to formulae 3 and 4 which, in the intestine, in particular in the ileum, specifically cause a blockade of bile acid resorption.

Inhibitors of bile acid resorption within the meaning of the invention are taken to mean substances which prevent the reuptake of bile acids in the intestine/ileum via a receptor-mediated process. These are, in particular, benzothiazepine derivatives (U.S. Pat. No. 5,998,400, U.S. Pat. No. 6,277,831), benzothiepine 1,1-dioxide derivatives (U.S. Pat. No. 6,221,897, WO 97/33882), in particular compounds according to formulae 3 and 4 which, in the intestine, in particular in the ileum, specifically cause a blockade of bile acid resorption. 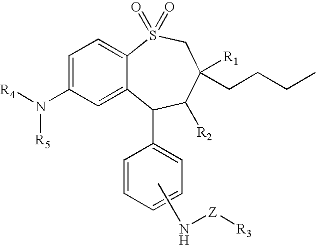
Formula 3: Benzothiepine Derivatives
(where R═C₆H₄NHZR₃; R¹, R⁴, R⁵=Me, Et, Pr, Bu; R²=H, OH, NH₂, amino(alkyl); R³=sugar radical; Z=—(C═O)ₙ-(C₀-C₁₆)-alkyl-, —(C═O)ₙ—(C₀-C₁₆)-alkyl-NH—, —(C═O)ₙ—(C₀-C₁₆)-alkyl-O—, —(C═O)ₙ—(C₀-C₁₆)-alkyl-(C═O)ₘ or a covalent bond; n=0 or 1; m=0 or 1, and also salts thereof) 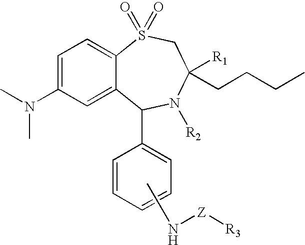
Formula 4: Benzothiazepine Derivatives
(where R¹=Me, Et, Pr, Bu; R²═H, OH; R³=sugar radical; Z=—(C═O)ₙ—(C₀-C₁₆)-alkyl-, —(C═O)ₙ—(C₀-C₁₆)-alkyl-NH—, —(C═O)ₙ—(C₀-C₁₆)-alkyl-O—, —(C═O)ₙ—(C₀-C₁₆)-alkyl-(C═O)ₘ or a covalent bond; n=0 or 1; m=0 or 1, and also salts thereof)

Cholesterol absorption inhibitors are active substances which inhibit in the intestine the receptor-mediated transport of cholesterol and thus increase the excretion of cholesterol, which finally leads to a moderate reduction of the serum cholesterol level. These include, in particular, hydroxy-substituted azetidinone cholesterol absorption inhibitors of the group 1-(4-fluorophenyl)-3(R)-[3(S)-(4-fluorophenyl)-3 hydroxypropyl)] 4(S) 4 hydroxyphenyl) 2 azetidinone) and 1-(4-fluorophenyl)-3(R) [3(R) (4 fluorophenyl)-3 hydroxypropyl)]-4(S) 4-hydroxyphenyl)-2-azetidinone) and their pharmacologically active salts or else substituted β-lactam cholesterol absorption inhibitors (e.g. WO-A-95/35277, WO-A-02/058733, WO-A-02/50060).

The group of the fibrates includes, inter alia, clofibrate, etophyllinclofibrate, bezafibrate, ciprofibrate, clinofibrate, binifibrate, lifibrole, fenofibrate, gemfibrozil, or etofibrate. Depending on the clinical picture, fibrates have a moderately reducing action on LDL cholesterol with a slight improvement of the HDL cholesterol values. Serum triglycerides are more strongly influenced by fibrates.

Nicotinic acid derivatives within the meaning of the invention are natural or synthetically prepared nicotinic acid, its esters or synthetic derivatives, for example niceritrol, nicofuranose, β-pyridylcarbinol or acipimox. This group of substances has a moderate effect on total and LDL cholesterol with simultaneously improved HDL cholesterol levels.

Phytosterols, within the meaning of the invention, are taken to mean 4-dimethylsterols, 4-monomethylsterols and 4,4-dimethylsterols and the respective esters and also plant extracts, mixtures and foods rich in phytosterols. These comprise β-sitosterol, campesterol, stigmatosterol, brassicasterol, desmosterol, chalinosterol, poriferasterol, clionasterol and all their natural or synthetic or isomeric derivatives. Plant stanols are taken to mean hydrogenated plant sterols, for example campestanol, sitostanol and the respective esters and also plant extracts, mixtures and foods rich in plant stanols.

Further plant extracts having a cholesterol-reducing activity include, inter alia, artichoke extracts and extracts of garlic and guglipid. They have already long been used as natural healing substances and exhibit moderate activity on the total and LDL cholesterol levels.

Guglipid (CAS 39025-24-6) within the meaning of the invention is the plant exudate of Commiphora mukul. (also Commiphora wightii or Balsamodendron mukul), a tree-like plant of the Burseraceae family. Guglipid within the meaning of the invention is likewise the “Guggulu”, “Guggul”, “Arka Guggalu” or “Gum Guggul” used in aryuvedic medicine. In addition, guglipids within the meaning of the invention are the extracts isolated from the plants of the Burseraceae family, or the isolates or pure substances isolated therefrom. Guglipids within the meaning of the invention are also the guggulsterols and isomers thereof, for example Z-guggulsterol (CAS 85769-67-1), guggulsterol I (CAS 39025-25-7), guggulsterol II (CAS 39025-26-8), guggulsterol III (CAS 39025-27-9), guggulsterol IV (CAS 20281-70-3), guggulsterol V (CAS 6120-71-4), guggulsterol VI (CAS 61391-01-3), 16-epiguggulsterol III (CAS 84709-26-2), E-guggulsterol, M-guggulsterol, dihydroguggulsterol-M, gugulsterol-Y and also guggulsterones. In addition, guglipids within the meaning of the invention are all plant sterols and stanols found in the plants of the Burseraceae family, in particular sitosterol, stigmasterol, cholesterol, campesterol and α-spinasterol. In addition, guglipids within the meaning of the invention are pharmaceutical products which are produced from the plant exudate or the pure chemical compounds, for example “gugulipid” from the company Legere Pharmaceuticals or food supplements, or food additives, for example “CholestGar” from the company Planetary Formulas.

Soy-protein-containing products within the meaning of the invention are taken to mean foods or food ingredients which consist of whole soybeans or have been produced from such, but also those which comprise processed soy protein products. These comprise, in particular, soy protein isolates, soy protein concentrates, soy flours, textured soy proteins (TSP) or textured vegetable proteins (TVP). In addition to the protein content, these food and food ingredients can also comprise naturally occurring soybean components, such as isoflavones, dietary fibers and saponins.

The inventive agents comprise at least one dietary fiber and at least one cholesterol-reducing active ingredient. In addition, the cholesterol-reducing agents can comprise conventional additives such as solvents, fillers, carriers such as methylcellulose, sweetening carbohydrates and other sweeteners, aromas, antioxidants etc. The combination of dietary fiber, in particular carob fiber, and active ingredients can also be administered in the form of two separate administration forms. Customary food applications such as bakery products, cereals, snacks or fruit bars, or drinks powders are suitable for dietary fibers, in particular carob fibers. Furthermore, the direct addition of the dietary fiber to self-produced foods and also use in food supplements of typical form (inter alia tablets, dragees, capsules, sachets, granules, bars etc.) is also possible, while the active ingredients are rather administered in typical manner in drugs (inter alia tablets, dragees, capsules, sachets, granules etc.).

A further preferred embodiment of the invention are agents which comprise a combination of carob fibers and levans as dietary fiber component.

The inventive agents comprise the active ingredients in amounts which are required to achieve a therapeutic effect in the case of administration 2 to 3 times per day. The dietary fiber component and, preferably, the carob fibers are likewise present in the inventive agents at concentrations which cause a marked cholesterol reduction. The daily dose of dietary fiber can be in the range from 1 to 50 g, customarily from 1 to 25 g, preferably from 5 to 15 g, and particularly preferably from 5 to 10 g. It is used in these amounts in combination with the usual daily doses of the active ingredients if a particularly extensive reduction of the cholesterol level is sought. For the active ingredient concentrations previously necessary for individual use, the concentrations in use can be reduced by up to 90% owing to synergies. Additives present if appropriate can be added at concentrations expediently of from 1 to 90% by weight, in particular from 10 to 60% by weight (based on the respective preparation form).

To produce the inventive agents, a procedure is best followed such that the desired amounts of dietary fiber and active ingredient are mixed with one another, spray dried, freed from solvent, agglomerated and/or instantized. In addition, all customary food technological and also pharmaceutical production processes such as pressing, kneading or dragee-coating can also be used.

In the combined administration according to the present invention, it has been found that the combined intake of dietary fiber, in particular carob fibers, and cholesterol-reducing active ingredients, lead to a markedly greater reduction of the cholesterol level than the sum of the effects in the case of administration of the individual components. It is surprising here that the additional administration of dietary fiber, in particular of carob fiber or levan, to the active ingredients, do not reduce the activity of the active compounds by non-specific interference, but that the observed effects go markedly beyond the effects achievable in the case of individual administration of the two substances.

The inventive agents thus permit a therapeutically frequently desirable greater reduction of the cholesterol level than was previously achievable, or effects at the same magnitude, but using lower amounts of active ingredient. They thus represent a significant advance in drug therapy of hypercholesterolemia or hyperlipidemia.

The inventive agents are expediently introduced in a suitable preparation matched to the most effective quantitative ratios. Suitable preparations for this are, for example, pulverulent or tablet-form preparations for dissolution, but also chewing tablets. These preparations can in addition comprise further ingredients (additives) to improve the dissolution, such as soluble carriers, tablet disintegrants, for example starch, cellulose, bentonite, pectin or peroxides and carbonates in combination with organic acids and generally colors, sweeteners such as sucrose, glucose, fructose and other carbohydrates, sugar alcohols such as sorbitol, xylitol, maltitol and Isomalt, or sweeteners, for example acesulfame K, cyclamate, saccharin, sucralose or aspartame and, in particular, aroma substances to improve acceptance.

The inventive agents may also be administered, however, separately in the form of a drug preparation of the active ingredient, and of the dietary-fiber-containing food or food supplement. For the active ingredient, customary drug administration forms such as tablets, capsules, solution for intake as drops or pulverulent preparation to be dissolved, or granules come into consideration. In this combined therapy, a suitable dietary fiber-containing food is in principle any food in which the dietary fiber can be incorporated, limits resulting from the properties of the food component and of the dietary fiber, as also from the intended application. Particularly suitable food would therefore be cereal-based foods such as bakery products, cereals, snacks and fruit bars, desserts, especially diet preparations such as drinks and, in particular, powdered drinks based on milk, fruit concentrates or fruit powders, carbohydrates or sugar alcohols. In the case of phytosterols and plant stanols, in addition, fat-containing foods come into consideration, for example spreadable vegetable fats, dressings and milk products.

The inventive agents may in addition be used as ingredient in animal nutrition or as feeds.

The invention will be discussed hereinafter with reference to examples.

### EXAMPLE 1

Determination of the Hypocholesterolemic Activity of Carob Fiber and Statins In Vivo

Hamsters are seen as a suitable animal model for propounding the present invention, even if the metabolic processes in hamsters and humans differ slightly. At all events, the two substances tested here in combination each give alone in humans a reducing effect on the serum cholesterol values, in particular on LDL cholesterol. The effect of a combined administration of carob fiber and a statin, here simvastatin, in this model should therefore also give conclusions for humans.

Male Syrian hamsters (100-120 g at the start of the study) received feed enriched with 0.35% cholesterol. The test substances carob fiber, produced by the method according to EP-A-0 616 780, and the statin simvastatin were mixed into the feed alone or in combination. The hamsters were divided into groups of 9 animals and treated with the test substances over a period of 28 days. After the animals were anesthetized, blood was obtained for determining the serum cholesterol values. The serum cholesterol contents were determined after obtaining the plasma from whole blood using a commercially obtained enzyme test kit. The total cholesterol content of the test groups thus determined were compared with the results of a control group which received no test substances. The results were as follows:

Results:

| | Total cholesterol | |
|---|---|---|
| | in blood serum | Changes from the |
| Treatment | (mmol/l) | control in % |
| Control | 7.65 | — |
| Carob fiber 2.5% | 7.17 | 6 |
| Simvastatin 1.5 mg % | 6.50 | 15 |
| Carob fiber 2.5% + | 5.73 | 25* |
| simvastatin 1.5 mg % | | |

*Synergy based on the total of the individual effects: +19%

### EXAMPLE 2

Determination of the Hypocholesteremic Activity of Carob Fiber and Phytosterols In Vivo

This experiment was carried out in a similar manner to Example 1. Instead of the simvastatin, margarine containing phytosterols was mixed into the hamster feed. The final concentration of the phytosterols in the feed was 0.5%.

Results:

| | Total cholesterol | |
|---|---|---|
| | in blood serum | Changes from the |
| Treatment | (mmol/l) | control in % |
| Control | 8.55 | — |
| Carob fiber 2.5% | 7.95 | 7 |
| Phytosterols 0.5% | 7.09 | 17 |
| Carob fiber 2.5% + | 6.16 | 28* |
| phytosterols 0.5% | | |

*Synergy based on the total of the individual effects: +17%

The possibilities of use of the inventive agents are explained by way of example by the following combined preparations:

### EXAMPLE 3

Pulverulent Preparation (for One Portion Size)

| | | |
|---|---|---|
| simvastatin | 5 | mg |
| carob fiber | 3 | g |
| xanthan (stabilizer) | 150 | mg |
| vanillin | 15 | mg |

Suspend the preparation in 150 ml of warm milk by stirring, and drink.

### EXAMPLE 4

Chewing Tablet

| | | |
|---|---|---|
| Vegapure ® 50 TP | 400 | mg |
| (phytosterol ester, Cognis Nutrition & Health, Germany) | | |
| carob fiber | 2 | g |
| sorbitol | 1.1 | g |
| magnesium stearate | 15 | mg |
| acesulfame K | 12 | mg |
| aspartame | 12 | mg |

| | |
|---|---|
| chocolate aroma | q.s. |

The chewing tablets are mixed and pressed in a conventional manner.

### EXAMPLE 5

Pulverulent Preparation (for One Portion Size)

| | | |
|---|---|---|
| lovastatin (MSD Sharp and Dome GmbH, D-85540 Haar) | 10 | mg |
| levan | 3 | g |
| xanthan (stabilizer) | 150 | mg |
| vanillin | 15 | mg |

Suspend the preparation in 150 ml of warm milk by stirring and drink.

1. A cholesterol-reducing agent comprising at least one dietary fiber selected from the group consisting of carob fruit flesh, a product isolated from carob fruit flesh or levan and at least one cholesterol-reducing active ingredient, except for a combination of a) a dietary fiber and b) an aryl-substituted propanolamine derivative or 1,4-benzothiepine 1,1-dioxide derivative.

2. The cholesterol-reducing agent as claimed in claim 1, wherein the dietary fiber or dietary fibers are present in a daily dose of from 1 to 50 g.

3. The agent as claimed in claim 1, wherein, in addition to carob fruit flesh, a product isolated from carob fruit flesh or levan, one or more dietary fibers from one or more of the following substances are present: whole grain cereals, oat bran, β-glucan, rice bran, corn bran, barley, Psyllium, guar, carob beans, tragacanth, pectin, inulin, indigestible oligosaccharides, linseed, soy dietary fiber, soy bran, dextrins, arabinoxylans and arabinogalactans.

4. The agent as claimed in claim 1, wherein the dietary fiber is carob fiber.

5. The agent as claimed in claim 1, wherein the dietary fiber is insoluble in water.

6. The agent as claimed in claim 1, wherein the active ingredient is selected from one or more of the following substances: statins, inhibitors of bile acid resorption, bile acid sequestrants, fibrates, nicotinic acid derivatives, phytosterols, plant stanols, cholesterol-reducing plant extracts, gugulipid and soy protein-containing products.

7. A cholesterol-reducing combination preparation comprising at least one dietary fiber selected from the group consisting of carob fruit flesh, a product isolated from carob fruit flesh or levan and at least one cholesterol-reducing active ingredient, except for an aryl-substituted propanolamine derivative or 1,4-benzothiepine 1,1-dioxide derivative, wherein said dietary fiber and said cholesterol-reducing active ingredient are present in separate administration forms.

8. The cholesterol-reducing combination preparation as claimed in claim 7, wherein, in addition to carob fruit flesh, a product isolated from carob fruit flesh or levan, one or more dietary fibers from one or more of the following substances are present: whole grain cereals, oat bran, β-glucan, rice bran, corn bran, barley, Psyllium, guar, carob bean seeds, tragacanth, pectin, inulin, indigestible oligosaccharides, carob fruit flesh or a product isolated from carob fruit flesh, linseed, soy dietary fiber, soy bran, dextrins, arabinoxylans and arabinogalactans.

9. The cholesterol-reducing combination preparation as claimed in claim 7, wherein the dietary fiber or the dietary fibers is (are) present in a daily dose of from 1 to 50 g, and the at least one cholesterol-reducing active ingredient is present in separate administration forms.

10. The cholesterol-reducing combination preparation as claimed in claim 7, wherein one or more of the dietary fibers are foods.

11. The cholesterol-reducing combination preparation as claimed in claim 7, wherein the cholesterol-reducing active ingredient is a food or drug.

12. A method for producing an agent as claimed in claim 1, wherein at least one dietary fiber and at least one cholesterol-reducing active ingredient are mixed with one another.

13. A drug comprising an agent as claimed in claim 1.

14. A drug as claimed in claim 13 wherein said drug is a cholesterol-reducing drug.

15. A drug claimed in claim 13 wherein said drug is a hypercholesterolemia, hyperlipidemia or arteriosclerosis reducing drug.

16. A food or a food ingredient comprising an agent as claimed in claim 1.

17. A food or a food ingredient as claimed in claim 16, wherein said food or food ingredient is a cholesterol-reducing food or food ingredient.

18. A drug comprising a combination preparation as claimed in claim 7.

19. A drug as claimed in claim 18 wherein said drug is a cholesterol-reducing drug.

20. A drug as claimed in claim 18 wherein said drug is a hypercholesterolemia, hyperlipidemia or arteriosclerosis-reducing drug.

21. An animal feed comprising an agent as claimed in claim 1.

22. A feedstuff comprising an agent as claimed in claim 1.
